(19) United States
(12) Patent Application Publication
(10) Pub. No.: US 2020/0319192 A1
(43) Pub. Date: Oct. 8, 2020

(54) EXTRACTION OF CIRCULATING TUMOR CELLS

(71) Applicant: INEMBER AB, Ytterby (SE)

(72) Inventor: Andreas JOSEFSSON, Ytterby (SE)

(21) Appl. No.: 16/303,644

(22) PCT Filed: May 24, 2017
(86) PCT No.: PCT/EP2017/062676

§ 371 Date: Nov. 20, 2018

(30) Foreign Application Priority Data
May 26, 2016 (SE) 1650729-5

(51) Int. Cl. G01N 33/574; G01N 33/543; G01N 33/50; C12Q 1/6886; A61K 33/243; A61K 31/337; A61K 31/704; A61K 31/135; A61K 31/436

(52) U.S. Cl. CPC G01N 33/57492; G01N 33/54326; G01N 33/57434; G01N 33/5091; C12Q 1/6886; A61K 33/243; G01N 2333/705; A61K 31/704; A61K 31/135; A61K 31/436; G01N 2800/52; C12Q 2600/158; A61K 31/337

(57) ABSTRACT

The present disclosure relates to a method of extracting circulating tumor cells (CTCs) from a blood sample from a human subject. This method includes adding a hyaluronan degrading enzyme to a blood sample from a subject in order to remove hyaluronan from the surface of the CTCs, bringing a CTC-capturing agent in contact with the CTCs and separating captured CTCs from the blood sample.

### TECHNICAL FIELD

The present invention relates to a method of extracting circulating tumor cells (CTCs) from a blood sample of a human subject. The invention further relates to a method of diagnosing cancer in a human subject as well as to a method of predicting the outcome of cancer therapy in a subject.

### BACKGROUND

Circulating tumor cells (CTCs) are cells which originate from either a primary tumor or a metastasis and which have shed into the vasculature and circulate in the bloodstream. CTCs may thus be seen as seeds for secondary tumor formation in vital distant organs. Secondary tumors often go undetected and are responsible for at least 90% of cancer-related deaths (Hayashi et al. Breast Cancer 2012, 19, 110-117; Mehra et al. Clin Cancer Res. 2015, 21, 4992-5). The presence of CTCs in blood may indicate approaching, or ongoing metastasis, or that an ongoing treatment is ineffective. Methods for detecting and analyzing CTCs may thus be highly valuable. Cancer therapies in metastatic disease are often targeted for specific gene alterations or molecular pathways and the characterization of the metastasis is then needed. The isolation of CTCs has been proposed to constitute a “wet biopsy” of the metastasis and might therefore serve as a more easily accessible and representative sample of the metastasis, for example when a specific target for cancer therapy is sought.

CTCs may be of different phenotypes, some more aggressive than others, some susceptible to a certain treatment, some resistant. The characterization of CTCs with regard to their phenotype may thus be helpful for decisions related to types of treatment etc. Different CTC-methods show different results when CTCs are extracted from the same patient. This suggests that different phenotypes are more easily extracted. Biomarkers associated with more aggressive phenotypes are genes involved in, for example, epithelial-mesenchymal transition, mesenchymal stem cells, resistant mechanism and cellular pathways related to metastatic potential. Biomarkers for characterization of CTCs, for example AR-V7, ALDH, TWIST1, EGFR, AKR1C3, MYC, ANXA2R and AURKA, are included in commercially available gene expressions panels. AR-V7 is a consecutively active AR shown to predict primary resistence to second line AR-signaling inhibitor (Antanorakis et al., N Engl J Med, 2014, 371(11): p. 1028-38). ALDH (aldehyde dehydrogenase gene) has in previous studies been shown to be associated with tumor initiating cells and with higher probability of forming metastasis and has been proposed to be a marker of more aggressive phenotypes (van den Hoogen, C., et al., Cancer Res, 2010. 70(12): p. 5163-73). TWIST1 has been shown to be upregulated in cancer cells with higher potential of metastatic spread (Chang, Y. S., et al. Cancer Res, 2015. 75(15): p. 3077-86). SPINK1 (Serine protease inhibitor Kazai-type 1) upregulation represent an aggressive phenotype in prostate cancer (Attard, G. et al. Lancet, 2015, 387(10013): p. 70-82,). ANXA2R (Annexin A2 receptor) has been shown to regulate adhesion, migration and homing of prostate cancer (Shiozawa, Y., et al. J Cell Biochem, 2008, 105(2), p. 370-80). AURKA (aurora kinase A) could be a driver of epithelial-mesenchymal-transition and therefore also to a more aggressive phenotype (Nouri, M., et al. Frontiers in oncology, 2014. 4: p. 370).

Methods applicable to CTC characterization include methods for DNA or mRNA characterization e.g. fluorescence in situ hybridization, AdnaTest and PCR-based methods and protein identification methods such as western blot or LC-MS.

Detection, enumeration as well as characterization of CTCs is difficult due to the low concentration of CTCs in blood. To improve the possibility to detect CTCs, methods for enrichment or isolation of CTCs have been developed and include for example density-gradient centrifugation, filtration and immunomagnetic separation. It has been discovered that a significant number of CTCs are masked by cells, protein, biomolecules and other factors, shielding the CTCs from surface interactions as an effective immune escape mechanism. Methods for revealing masked CTCs are disclosed in WO 2011/028905.

There is, however, still a need in the field of cancer diagnosis for improved CTC detection methods.

### DESCRIPTION OF THE INVENTION

One object of the invention is to at least in part alleviate the drawbacks of the prior art with respect to extraction of circulating tumor cells, and, in turn, the detection of circulating tumor cells.

According to one aspect of the invention, there is provided a method of extracting circulating tumor cells (CTCs) from a blood sample from a human subject, comprising the steps of:
a. adding a hyaluronan degrading enzyme to a blood sample from a subject in order to remove hyaluronan from the surface of said CTCs;
b. bringing a CTC-capturing agent in contact with said CTCs; and
c. separating captured CTCs from said blood sample.

In other words, there is provided a method of extracting circulating tumor cells (CTCs) from a blood sample from a human subject. Said blood sample is suspected to contain CTCs. A hyaluronan degrading enzyme is added to the blood sample which has been previously obtained from a human subject. Addition of a hyaluronan degrading enzyme removes hyaluronan from the surface of said CTCs. A CTC-capturing agent is then brought into contact with said CTCs, and any captured CTCs are extracted from the blood sample.

Hyaluronan, also called hyaluronic acid, is a polysaccharide, more specifically a non-sulphonated glucoseaminoglycan with the repeating unit→4)-β-GlcA-(1→3)-β-GlcNAc-(1→, with all sugars in the D pyranoside configuration i.e a polymer of disaccharides composed of D-glucoronic acid and D-N-acetylglucoseamine.

In one embodiment, the method may further comprise an initial step of providing a blood sample from a human subject.

According to previous known methods for extraction, isolation or enrichment of circulating tumor cells, a CTC-capturing step based on recognition of the CTCs is often employed. CTCs, however, are sometimes masked, some phenotypes more than others, by hyaluronan, a glucoseaminoglycan. The consequence is that CTC-capturing agents based on molecular recognition may also fail to identify and capture the CTC. According to the methods as disclosed herein, by the addition of a hyaluronan degrading enzyme to a blood sample comprising CTCs masked with hyaluronan, the hyaluronan is removed or degraded, at least in part. This results in less hyaluronan being bound to the CTCs, making molecular recognition of CTC surface molecules more effective. It has been found that by demasking the CTCs using a hyaluronan degrading enzyme, the fraction of CTCs that are detectable increases, and more CTCs may be for example enumerated or characterized as shown in the appended examples. Further, sub-populations of CTCs not being detectable by conventional methods may, by making use of the method of extracting CTCs as disclosed herein, be extracted and analyzed. Some especially aggressive phenotypes are difficult to isolate due to the hyaluronan on their surface. Aggressive phenotypes may express, for example, both AR-V7 inside the cell and hyaluronan on the surface of the CTCs and may therefore be difficult to isolate using e.g. immunomagnetic separation. However, by use of the method as disclosed in the first aspect, comprising addition of a hyaluronan degrading enzyme, such aggressive phenotypes may to a larger extent be extracted and subsequently detected.

It is to be understood that isolation and extraction may be used interchangeably.

The extraction of CTCs according to the invention enables the detection of a larger number of unique genes compared to a CTC extraction method that does not include the incubation of the blood sample with hyaluronidase, as shown in Example 4. The genes AGR2, AHR, AKT2, ALDH, ANXA2R, AR, ARV7, BCL2, CDH1, CDH2, EGFR, EMP2, HER2, KLK, KRT19, POU5F1, PTCH1, RUNX2, SNAI1, SPINK1, TACSTD2, TUBB3 and TWIST1 were detected in blood samples only when the extraction of the CTCs included incubation with hyaluronidase.

The method of the invention increases sensitivity with regard to identifying tumor specific genes in CTCs, by degrading hyaluronan in the blood samples before extractions of the CTCs, which is shown in both parallel (example 4) and consecutive studies, see example 2 and 3.

The method according to the invention increases the number as well as amount of isolated CTCs, by degrading hyaluronan in the blood sample before extraction of CTCs, as shown in example 1 and 4, respectively.

In one embodiment of this aspect, the hyaluronan degrading enzyme is a hyaluronidase.

It is to be understood that the term hyaluronan degrading enzyme means any type of enzyme capable of degrading hyaluronan.

It is to be understood that the term hyaluronidase means any type of hyaluronidase if not specified otherwise. Examples of hyaluronidases include hyaluronate glycanohydrolase (EC 3.2.1.35), hyaluronate glycanohydrolase (EC 3.2.1.36), hyaluronate lyase (EC 4.2.99.1).

In one embodiment of this aspect, the hyaluronidase is selected from hyaluronate glycanohydrolase (EC 3.2.1.35), hyaluronate glycanohydrolase (EC 3.2.1.36), hyaluronate lyase (EC 4.2.99.1).

In one embodiment of this aspect, the hyaluronidase is specifically hyaluronate glycanohydrolase (EC 3.2.1.35).

In one embodiment of this aspect, the CTC-capturing agent comprises a molecular moiety having affinity for a molecular moiety on the surface of a CTC. The CTC capturing agent may for example comprise a protein or polypeptide having affinity for a molecular moiety on the surface of a CTC, for example for a protein expressed on the surface of said CTC.

In one embodiment of this aspect, the CTC-capturing agent comprises an antigen binding moiety, said moiety having affinity for a membrane-bound antigen of said CTCs.

According to one embodiment of this aspect of the invention, the antigen binding moiety is an antibody. The specific nature of an antibody-antigen interaction makes an antibody a suitable molecule for binding a membrane-bound antigen on CTC.

To facilitate separation, the CTC capturing agent may be immobilized. The CTC capturing agent may for example be immobilized on a surface, such as the surface of a tube or well.

In one embodiment, the antigen binding moiety is immobilized on a magnetic bead. Magnetic beads coated with an antigen binding moiety may be used to first capture or bind CTCs and subsequently to retrieve the captured CTCs by use of a magnet. In such an embodiment, an antigen binding moiety immobilized on a magnetic bead may be referred to as a CTC capturing agent.

According to one embodiment of this aspect of the invention, steps b and c are realized by immunomagnetic separation. Immunomagnetic separation is a commonly used technique for CTC isolation.

According to one embodiment of this aspect of the invention, the membrane-bound antigen is a transmembrane glycoprotein.

According to one embodiment of this aspect of the invention, the membrane-bound antigen is selected from epithelial cell adhesion molecule (EpCAM) and human epidermal growth factor receptor 2 (HER2).

EpCAM is used as a diagnostic marker for various cancers. Moreover, it appears to play a role in tumorigenesis and metastasis of carcinomas, so it can also function as a potential prognostic marker as well as a potential target for immunotherapy. Although EpCAM was initially described as a dominant surface antigen on human colon carcinoma, it has been found to be a diagnostic marker for various types of cancer, including prostate cancer and colon cancer. Since EpCAM is a common marker for various cancers, antibodies for EpCAM may be used in the detection and isolation of CTCs. Anti-EpCAM antibodies are commercially available.

HER2 is an oncogene and a member of the human epidermal growth factor receptor (HER/EGFR/ERBB) family. In tumor cells, oncogenes are often mutated or expressed at high levels. Amplification or overexpression of HER2 has been shown to be involved in the development and progression of certain aggressive types of breast cancer. Antibodies or other molecular moieties targeting HER2 may thus be used in the detection and isolation of CTCs and are commercially available (anti-HER2).

According to one embodiment of this aspect of the invention, the membrane-bound antigen is epithelial cell adhesion molecule (EpCAM). CTCs of prostate cancers have to a varying extent bound hyaluronan hiding the CTCs from anti EpCAM antibodies. As disclosed above, EpCAM is a surface antigen on CTCs occurring in subjects having, for example, prostate cancer. The use of hyaluronan degrading enzyme may make EpCAM on CTCs more accessible for CTC isolation.

Incubation of blood samples with hyaluronidase provides CTC that are more accessible for CTC isolation using anti-EpCAM and subsequently counted and/or characterized.

According to one embodiment of this aspect of the invention, step a further comprises incubating the blood sample comprising the hyaluronan degrading enzyme at a temperature of from 8 to 40° C., preferably from 15 to 37° C., more preferably from 18 to 25° C. Incubating a blood sample which may comprise CTCs with a hyaluronan degrading enzyme may be done during a time period of from 5 to 50 minutes, preferably from 10 to 30 minutes, more preferably from 15 to 25 minutes.

According to one embodiment of this aspect of the invention, said captured CTCs belong to a sub-population of CTCs.

According to one embodiment of this aspect of the invention, at least some of said captured CTCs belong to a sub-population of CTCs. The captured CTCs according to the invention includes both a sub-population of CTCs accessible to isolation and detection without prior addition of a hyaluronan degrading enzyme as well as a sub-population not accessible to isolation or detection without the addition of a hyaluronan degrading enzyme. The sub-population not accessible to isolation or detection without the addition of a hyaluronan degrading enzyme may be isolated and detected either after a prior isolation of CTC without the hyaluronidase incubation or without a prior isolation of CTC without the hyaluronidase incubation or without.

According to one embodiment of this aspect of the invention, the sub-population is not detectable without the addition of a hyaluronan degrading enzyme.

According to one embodiment of this aspect of the invention the sub-population of CTCs are of an especially aggressive phenotype. For example the sub-population of captured CTCs express to a higher extent the resistant mechanisms AR-V7.

According to one embodiment of this aspect of the invention, the captured CTCs to a higher extent expresses detectable levels of genes selected from AR-V7, AKR1C3, AKT2, ALDH1A1, AURKA, BCL2, CDH1, KLK3, SPINK1 and TOP2A.

According to one embodiment of this aspect of the invention, said sub-population of CTCs expresses to a higher extent genes selected from AR-V7, AKR1C3, AKT2, ALDH1A1, AURKA, BCL2, CDH1_1, KLK3, SPINK1 and TOP2A.

According to one embodiment of this aspect of the invention, the human subject has, or is susceptible of having, cancer. A primary tumor or metastasis may release tumor cells circulating into the blood stream. Subjects that have been diagnosed with cancer may develop secondary tumors or metastases, even though the primary tumor has been treated. The finding of extracted CTCs may be an early indication of possible secondary tumors. Cancers which utilize CTCs as a means for spreading may use CTCs masked with hyaluronan. Such CTCs may be demasked by incubation with a hyaluronan degrading enzyme as disclosed herein. Such cancers include breast cancer, bladder cancer, colorectal cancer, ovarian cancer, melanoma and prostate cancer.

According to one embodiment of this aspect of the invention, the cancer is selected from breast cancer, colorectal cancer, bladder cancer, ovarian cancer, melanoma and prostate cancer.

According to one embodiment of this aspect of the invention, the subject has or is suspected of having prostate cancer.

According to one embodiment of this aspect of the invention, the subject has or is suspected of having metastasized prostate cancer. A subject having a metastatic spread of cancer may have CTCs masked with hyaluronan in the blood. Extraction or isolation of such CTCs by use of the methods as disclosed herein, followed by e.g. enumeration and/or characterization of the CTCs, may be used to for example choose a suitable treatment of metastatic cancer. Previous methods for isolation may miss the hyaluronan masked CTCs, which are most crucial to find in metastasized cancer, and may thus result in an underestimated count and/or incorrect characterization of any CTCs. This may in turn result in an incorrect prediction of the outcome of a therapy and/or an incorrect diagnosis of the disease.

According to one embodiment of this aspect of the invention, the subject has or is suspected of having castration-resistant prostate cancer (CRPC). Most hormone dependent cancers become resistant to treatment after one to three years and resume growth despite castration therapy. The term castration-resistant refers to that they are no longer responsive to castration treatment or reduction of available androgens by chemical or surgical means. These cancers still show reliance upon androgen signaling, through different mechanisms of resistance to therapy, for example AR-V7 expression. Enumeration and/or characterization of CTCs from blood samples of CRPC patients help monitoring the development of the disease, predicting outcome of given treatment and give more information about the cancer, which may be helpful for selecting the right treatment. Previous methods for isolation may miss the hyaluronan masked CTCs, which are most crucial to find in CRPC patients, and may thus result in an underestimated count and/or incorrect characterization of any CTCs. This may in turn result in an incorrect prediction of the outcome of a therapy and/or an incorrect diagnosis of the disease.

According to one aspect of the invention, there is provided a method of
i) diagnosing cancer in a human subject, or
ii) predicting the outcome of cancer therapy in a subject,
said method comprising extracting circulating tumor cells according to the method of the first aspect of the invention, further comprising the step of:
d. analyzing the extracted CTCs.

Analysis of the extracted CTCs may involve for example enumeration or characterization. Characterizing CTCs with regards to different genes, mRNAs or phenotypes, may provide useful information about the cancer type and for selecting a treatment. Demasking CTCs using a hyaluronan degrading enzyme may provide an increased CTC count and also reveal sub-populations of CTCs having especially aggressive phenotypes that are difficult to detect due to the presence of hyaluronan on their surface. Phenotypes of CTC expressing a mechanism of resistance to androgen signaling targeted therapy, for example AR-V7, may be difficult to isolate using immunomagnetic separation using previous methods. The removal of hyaluronan on the CTC surface by incubation with for example hyaluronidase may unveil CTCs of phenotypes expressing such a resistance mechanism and enable the isolation or detection of such CTCs. Patients with CTC phenotypes expressing AR-V7 are resistant to newly developed androgen signaling targeted therapy, such as abiraterone acetate and enzalutamide, and would not respond to any of these treatments. The isolation and identification of CTCs of certain phenotypes may thus indicate that the cancer has resistance mechanisms to certain treatments and would benefit from an alternative treatment, such as chemotherapy. Thus, by employing the method according to the first aspect, CTC enumeration and/or characterization may improve, thereby enabling improved cancer diagnosis and/or prediction of the outcome of a particular cancer therapy.

In other words, the method according to the invention makes the isolation of CTCs more effective than a corresponding method without hyaluronidase incubation, and thus increases the sensitivity of a subsequent detection of genes selected for identifying targets for cancer therapy and/or resistance mechanism towards cancer therapy.

According to one aspect of the invention, there is provided a method of diagnosing cancer in a human subject, said method comprising using a method of extracting circulating tumor cells according to the invention, further comprising the step of:
d. analyzing the extracted CTCs.

According to one aspect of the invention, there is provided a method of predicting the outcome of cancer therapy in a subject, said method comprising using a method comprising extracting circulating tumor cells according to the invention, further comprising the step of:
d. analyzing the extracted CTCs.

According to one embodiment of the above aspects of the invention, the analyzing is selected from characterizing and counting said extracted CTCs. Thus, either the analysis is qualitative or quantitative.

According to one embodiment of the above aspects of the invention, the analyzing is characterizing the extracted CTCs. Such characterizing may be done by a method such as the AdnaTest Prostate Cancer Select protocol, such as AdnaTest Prostate Cancer Select protocol followed by AdnaTest Prostate Cancer Detect protocol.

According to one embodiment of the above aspects of the invention, the characterizing comprises detection of any androgen receptor splice variant. An example of an androgen receptor splice variant is AR-V7. Androgen receptor splice variants are associated with resistance to androgen deprivation therapy. Detection of androgen receptor splice variants with a method according to the invention may thus predict resistance to androgen deprivation therapy.

According to one embodiment of the above aspects of the invention, the cancer therapy of ii) is targeted cancer therapy.

According to one embodiment of the above aspects of the invention, the analyzing is counting the extracted CTCs. In other words, one embodiment of the above aspects of the invention, further comprises enumerating the extracted CTCs. Such enumeration may be done by the CellSearch method.

According to one aspect of the invention, there is provided a method of monitoring the response to cancer therapy, said method comprising a method comprising extracting circulating tumor cells according to the invention, further comprising the step of:
d. analyzing the extracted CTCs.

Monitoring the response to cancer therapy is a way to assess the efficacy of a cancer treatment. This may be done by comparing the CTC count before the start of a treatment and after an amount of time after initiating the treatment. By using a method according to the invention, a higher CTC count may be obtained, which may give a more reliable assessment.

According to one embodiment of this aspect of the invention, the analyzing is selected from characterizing and counting said extracted CTCs.

According to one embodiment of this aspect of the invention, the analyzing is characterizing the extracted CTCs.

According to one embodiment of this aspect of the invention, the characterizing comprises detection of any androgen receptor splice variant.

According to one embodiment of this aspect of the invention, the analyzing is counting the extracted CTCs.

According to one embodiment of this aspect of the invention, the cancer therapy is targeted cancer therapy.

According to one aspect of the invention, there is provided a method of finding a target for therapy, comprising performing the method according to the invention; characterizing the extracted CTCs with regard to expressed genes; and selecting at least one therapeutic target from genes expressed in the extracted CTCs.

According to one aspect of the invention, there is provided a method of treatment of a cancer in a human subject, said method comprising extracting circulating tumor cells according to the first aspect of the invention, further comprising the steps of:
d. analyzing the extracted CTCs;
e. selecting a therapeutic agent; and
f. administering a therapeutically effective amount of the therapeutic agent to a human subject in need thereof.

The selecting of a therapeutic agent is based on the result of the analysis in d.

According to one embodiment of this aspect of the invention, the analyzing is selected from characterizing or counting said extracted CTCs.

According to one embodiment of this aspect of the invention, the analyzing is characterizing the extracted CTCs. Analyzing in this context is typically characterizing with regards gene expression. Genes expressed correspond to potential therapeutic targets. Genes that were only detected using the extraction method of the invention are AGR2, AHR, AKR1C3, AKT2, ALDH1A1, AR, AURKA, BCL2, CDH1, EMP2, KLK3, SNAI1, SPINK1, SRD5A1, TOP2A, TWIST1, and VEGFA. Further, the genes AHR, AR, AKR1C3, AKT2, ALDH1A1, AURKA, BCL2, CDH1, SPINK1, TOP2A and VEGFA correspond to therapeutic targets to which there are already available FDA approved drugs and these were exclusively detected when using the method of extracting CTCs according to the invention.

According to one embodiment of this aspect of the invention, the characterizing comprises detection of any androgen receptor splice variant.

According to one embodiment of this aspect of the invention, the analyzing is counting the extracted CTCs.

According to one aspect of the invention, there is provided kit for enhancing the extraction circulating tumor cells (CTCs) from a blood sample from a human subject, comprising
a hyaluronan degrading enzyme;
a buffer with a pH of 5-9; and
instructions to use the kit with a CTC-capturing agent,
   wherein the kit is to be used in the method according to the invention.

Buffer solutions and buffering agents suitable for incubating the hyaluronan degrading enzyme are for example phosphate, N-(2-Acetamido)iminodiacetic acid, 2-[4-(2-hydroxyethyl)piperazin-1-yl]ethanesulfonic acid, tris-hydrochloride, 2-(cyclohexylamino)ethanesulfonic acid, Bis-Tris, MOPS, HEPES, citrate, acetate, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate buffer, calcium hydroxide phosphate, bicarbonate, sodium lactate, dibasic sodium phosphate, monobasic sodium phosphate, sodium phosphate mixtures, tromethamine, magnesium hydroxide, aluminum hydroxide and alginic acid. According to one embodiment of this aspect of the invention, the buffer is a phosphate buffer.

The buffer may contain 0.001-0.1 M of a buffering agent, such as 0.01-0.08 M.

According to one embodiment of this aspect of the invention, the buffer has a pH of 5.5-8.5, such as 6-8, such as 6.5-7.5, such as 7.

According to one embodiment of this aspect of the invention, the buffer further comprises serum albumin, such as 0.001-0.2% serum albumin, such as 0.005-0.1% serum albumin, such as 0.005-0.05% serum albumin. Typically the serum albumin is bovine serum albumin.

According to one embodiment of this aspect of the invention, the hyaluronan degrading enzyme is hyaluronidase and said buffer is a phosphate buffer with a pH of 6-8.

According to one embodiment of this aspect of the invention, the kit further comprises a CTC-capturing agent.

### BRIEF DESCRIPTION OF THE DRAWINGS

FIG. 1 is a chart showing changes in CTC count between hyaluronidase treated (E+) and control sample (E−), visualized as percentages, for each patient. Bars above the line have higher CTC count after hyaluronidase treatment and bars under the line have lower CTC count after hyaluronidase treatment. The mean count for each patient is denoted under the x-axis.

FIG. 2 is a chart showing the number of patients with uniquely detected genes in hyaluronidase treated samples (E+) and and control samples (E−) respectively. The positive bars show the number of patients with uniquely detected genes in E+ samples (grey and black bars) for each gene. Black bars represent the genes with at least 3 of the 15 patients showing uniquely detection of that gene in E+ samples, and the white bars show the number of men with uniquely detected genes in E− samples.

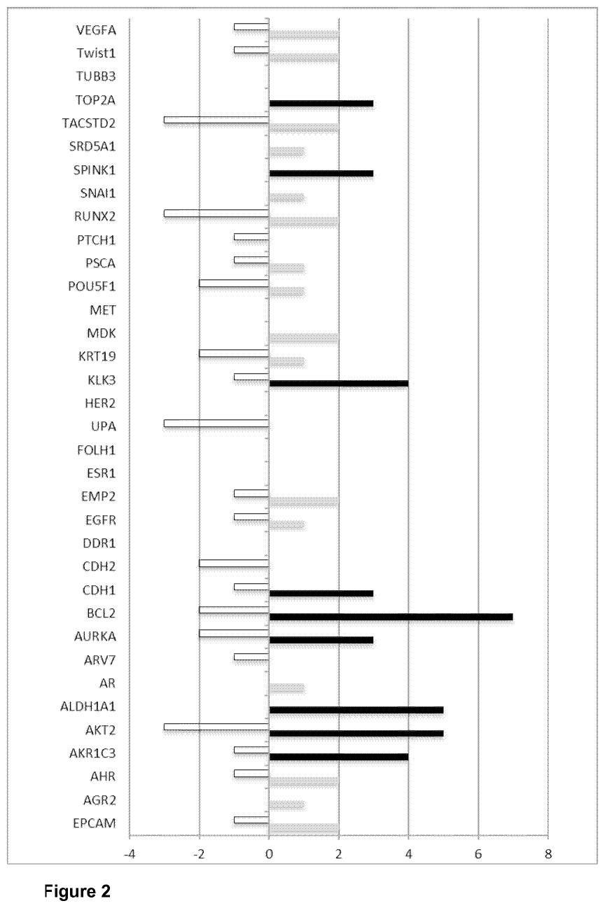

### EXAMPLES

The present invention will now be described in more detail by the following non-limiting examples.

### General Procedures

Hyaluronidase from bovine testes (EC #3.2.1.35) was purchased from Sigma Aldrich(Saint Louis, Mo.). The hyaluronidase in powder form was stored in −20° C. until used. The dilution buffer was prepared according to the manufactory instructions (0.02 M phosphate buffer with pH 7.0 with 77 mM NaCl and 0.01% bovine serum albumin) and was stored in 4-8° C. until used.

### Example 1

Evaluation with the Cellsearch© System

Men included in a prospective study of CTC as biomarker in metastatic prostate cancer were also included in this side study to find ways to increase the isolated CTC count.

13 patients with verified high risk prostate cancer were included in the study, out of which 2 patients analyzed on two separate occasions with at least 1 week apart. For each patient, the following procedure was followed. Three blood tubes were collected with the CellSave© tubes from the CellSearch© System. One was used as a reference sample (r), one as control (E−) and one as the enzymatic tube (E+). To the E+ tube, 5 mg Hyaluronidase, dissolved in 1 mL dilution buffer, was added. To the E− tube, 1 mL of dilution buffer was added. The E+ and the E− tubes were then incubated at room temperature for 20 minutes. The three samples were subsequently handled according to the CellSearch protocol (the FDA approved CellSearch system).

### Results

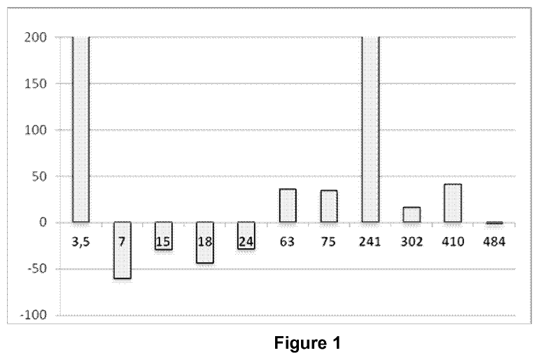

Two of the samples were negative with CellSearch system. The CTC change was larger between the E+ and E− compared to E− and r samples. The following results of the changes in E+ compared to E− and mean CTC count are shown in FIG. 1. The mean count for each patient in the analysis was 3,5 to 484. Of the 11 patients 2 did not have any CTC count in E− sample but 7 and 482 CTC after hyaluronidase treatment respectively (Bars going above 100%). The positive change of CTCs count between E+ and E− was more often seen in patients with higher mean CTCs. Accordingly 5 of the 6 patients with mean CTC count above 25 had higher CTC count in E+ compared to E−. In patients with lower than 25 CTC in mean only 1 of 5 patients had more CTC after E+ compared to E−. The results of those men with at least one CTC in any of the tests are shown in FIG. 1.

### Example 2

Evaluation with AdnaGene Select/Detect®

Blood was drawn from 4 men with high risk prostate cancer using AdnaCollect Blood Collection Tubes®. CTCs were extracted from each tube according to the AdnaTest ProstateCancer Select protocol. To the remaining blood (after extraction) was then added 5 mg Hyaluronidase dissolved in 1 mL dilution buffer and the mixture was incubated for 15 minutes at 37° C. A second extraction according to the AdnaTest Prostate Cancer Select protocol was then performed. mRNA was isolated and converted to cDNA and the CTC content was verified with a PCR-detection of PSA, PSMA and EGFR according to the manufactures instruction. An expression panel consisting of 48 genes related to prostate cancer metastasis and progression was used to analyze the gene expression pattern between the subsequent extractions of CTC with or without hyaluronidase incubation.

### Results

The gene expression panel showed that the extracted CTC after enzymatic treatment expressed characteristics of a more aggressive phenotype. AKR1C3, ALDH, AR-V7, EGFR, MyC, TWIST 1, ANXA2R and AURKA was all differentialy expressed in CTC with or without hyaluronidase incubation and with more detection of these genes after hyaluronidase incubation. The relative expression of the gene expression after hyaluronidase incubation compared to the expression without hyaluronidase incubation is shown in Table 1 for each patient and gene. In some cases the genes was only detected after hyaluronidase incubation and the relative expression is then shown as 999.

TABLE 1
Relative expression of qPCR detection with enzymatic incubation
compared to detection without enzymatic incubation
(999 = only detected after enzymatic incubation).

| Gene | Patient 1 | Patient 2 | Patient 3 | Patient 4 |
|---|---|---|---|---|
| AKR1C3 | 0 | 11 | 0 | 0 |
| ALDH | 999 | 1.8 | 0 | 2.8 |
| ARV7 | 0 | 999 | 0 | 0 |
| AURKA | 0 | 8 | 0 | 0 |
| EGFR | 0 | 999 | 0 | 0 |
| MyC | 0 | 4 | 0.7 | 2 |
| Twist1 | 999 | 2.3 | 0 | 0 |
| ANXA2R | 0 | 0.9 | 999 | 0 |

The study was approved by the local ethics committee.

### Example 3

Consecutive Isolation of CTC with AdnaGene Prostate Cancer Select/Detect from Blood Samples from Prostate Cancer Patients to Identify Genes Only after Hyaluronidase Incubation

Five patients before first line therapy and one patient before second line therapy of metastatic prostate cancer (including four of the patients of example 2) were included in this example. The procedure was the same as in example 2. One blood sample was collected from each patient using the AdnaCollect Blood Collection Tubes®. In summary two consecutive CTC isolations were performed from the same blood sample, the first of which was performed without Hyaluronidase treatment and the second with Hyaluronidase treatment. The first extraction of CTCs was performed according to the standard AdnaTest Prostate Cancer Select protocol. After the first extraction, 1 mL Hyaluronidase (5 mg/mL in dilution buffer) was added to the remaining blood and incubated for 15 minutes at 37° C. After incubation, the second extraction of CTCs was performed according to the AdnaTest Prostate Cancer Select protocol. CTC isolated from the first extraction is here on denoted as “standard-CTC” and the second isolation is denoted “enriched-CTC”.

### Characterization of the CTCs

Prostate cancer origin of the CTCs was confirmed by expression of PSA, PSMA, and EGFR detected by multiplex PCR, and semi-quantitatively analysed with the Agilent DNA 1000 Nano chip on an Agilent 2100 Bioanalyzer (Agilent Technologies, Santa Clara, Calif., USA), according to the AdnaGen Prostate Cancer Select protocol. To assess expression of a broader set of genes, 2 μl of cDNA samples (including beads from CTC collection) were pre-amplified using the TATAA PreAmp Primer Mix (TATAA Biocenter) and TATAA PreAmp GrandMaster® Mix (Cat. No. # TA05, TATAA Biocenter). Pre-amplification was performed in a thermocycler (T100, BioRad). Assays for the included genes (Table 2) were specially designed for this study (now available in the Grand Performance CTC Assay Panel (TATAA Biocenter) and ValidPrime™ assay (TATAA Biocenter). The qPCR was performed using TATAA Probe GrandMaster® Mix Low ROX (TATAA Biocenter) and GE 96.96 Dynamic Array™ Sample And Assay Loading reagent Kit (P/N 85000802-R, Fluidigm) and further by BioMARK (Fluidigm) using the 96-96 Dynamic Array™ IFC (Integrated Fluidic Circuit). All samples were analyzed in duplicates. No template-controls were included for the single assay qPCR.

TABLE 2
The genes included in the panel used
to characterize the isolated CTCs.

| | | | | |
|---|---|---|---|---|
| EPCAM | ARV7 | EMP2 | MDK | SPINK1 |
| AGR2 | AURKA | ESR1 | MET | SRD5A1 |
| AHR | BCL2 | FOLH1 | POU5F1 | TACSTD2 |
| AKR1C3 | CDH1 | UPA | PSCA | TOP2A |
| AKT2 | CDH2 | HER2 | PTCH1 | TUBB3 |
| ALDH1A1 | DDR1 | KLK3 | RUNX2 | TWIST1 |
| AR | EGFR | KRT19 | SNAI1 | VEGFA |

### Results

The genes detected in “enriched-CTC” (after the hyaluronidase incubation), showed characteristics of a more aggressive phenotype compared to “standard-CTC” (without prior hyaluronidase incubation). The results from the analysis are summarized in table 3. AR-V7 could be identified only after incubation with the enzyme both for patient number 2 (as previously shown in example 2) and also for patient number 5 (Table 3). The additional genes that was detected in the “enriched-CTC” in at least one patient are AGR2, AHR, AKR1C3, AKT2, ALDH1A1, AR, AURKA, BCL2, CDH1, EMP2, KLK3, SNAI1, SPINK1, SRD5A1, TOP2A, TWIST1, and VEGFA (Table 3). The three genes only detected in “standard-CTC” and not in “enriched-CTC” were ANXA2R, SRD5A1 and TOP2A. The mean “cycle of quantification” (Cq) value for EPCAM in “standard-CTC” and “enriched-CTC” respectively was not significantly different (p=0.35, two-sided student t-test). EPCAM expression is interpreted as a proxy for the amount of isolated CTC in the sample (since the antibody used for isolation in the AdnaGen-test is recognizing this epithelial antigen), and thus indicating that the amount of CTC isolated in second extraction (“enriched-CTC”) was not different form the first extraction. This result is surprising and may indicate that the CTCs detected after hyaluronidase incubation (“enriched-CTC”) were not available/accesible to for extraction in “standard-CTC”, and that “enriched-CTC” may have a different phenotype than “standard-CTC”.

TABLE 3
Summary of detection of expression in CTCs from consecutive
extractions of CTCs, before and after hyaluronidase incubation.
The table displays the detection frequency of the two extractions
for each patient and gene. “—” represents no
detection of the gene neither before nor after incubation,
“2” represents detection in both samples for that
patient, “1” represents detection in only “enriched-
CTC”, and “−1” represents detection only in
“standard-CTC”.

| | Patient 1 | Patient 2 | Patient 3 | Patient 4 | Patient 5 |
|---|---|---|---|---|---|
| EPCAM | 0 | 2 | 0 | 2 | 2 |
| AGR2 | 0 | 2 | 0 | 2 | 1 |
| AHR | 0 | 1 | 0 | 0 | 2 |
| AKR1C3 | 0 | 2 | 0 | 0 | 2 |
| AKT2 | 0 | 2 | 0 | 0 | 1 |
| ALDH1A1 | 1 | 2 | 0 | 2 | 2 |
| AR | 0 | 2 | 0 | 0 | 1 |
| ARV7 | 0 | 1 | 0 | 0 | 1 |
| AURKA | 0 | 2 | 0 | 0 | 2 |
| BCL2 | 0 | 1 | 0 | 0 | 1 |
| CDH1 | 0 | 2 | 0 | 0 | 1 |
| CDH2 | 0 | 0 | 0 | 0 | 1 |
| DDR1 | 0 | 0 | 0 | 0 | 0 |
| EGFR | 0 | 1 | 0 | 0 | 0 |
| EMP2 | 0 | 2 | 0 | 0 | 1 |
| ESR1 | 0 | 0 | 0 | 0 | 0 |
| FOLH1 | 0 | 2 | 0 | 2 | 2 |
| UPA | 0 | 0 | 0 | 0 | 2 |
| HER2 | 0 | 2 | 0 | 0 | 1 |
| KLK3 | 0 | 2 | 0 | 0 | 1 |
| KRT19 | 0 | 2 | 0 | 0 | 1 |
| MDK | 0 | 2 | 0 | 2 | 2 |
| MET | 0 | 0 | 0 | 0 | 0 |
| POU5F1 | 0 | 2 | 0 | 1 | 2 |
| PSCA | 0 | 2 | 0 | 0 | 0 |
| PTCH1 | 0 | 0 | 0 | 0 | 1 |
| RUNX2 | 0 | 0 | 0 | 0 | 1 |
| SNAI1 | 0 | 1 | 0 | 0 | 1 |
| SPINK1 | 0 | 0 | 0 | 2 | 1 |
| SRD5A1 | 0 | 2 | −1 | 0 | 2 |
| TACSTD2 | 0 | 2 | 0 | 1 | 2 |
| TOP2A | 0 | 2 | 0 | −1 | 2 |
| TUBB3 | 0 | 1 | 0 | 0 | 0 |
| TWIST1 | 1 | 2 | 0 | 0 | 1 |
| VEGFA | 0 | 2 | 0 | 0 | 2 |

### Example 4

### Hyaluronidase Incubation and CTC Isolation

CTCs were detected after hyaluronidase incubation in samples from 15 patients with prostate cancer with either high risk of metastatic prostate cancer or verified metastatic prostate cancer. The samples were taken before first or second line treatments and all patients had a PSA value above 100 ng/ml at sampling. In summary this experiment was performed by isolate CTC in parallel and pooled blood samples with either hyaluronidase incubation or control incubation with dilution buffer only. In more details the two blood samples were collected from each patient using EDTA-plasma tubes. The blood was refrigerated within 20 min and handled within 4 hours. Before CTC isolation the samples were pooled and then again divided in two tubes. They were further pre-treated by incubation with 5 mg Hyaluronidase dissolved in 1 mL dilution buffer (E+) or only 1 mL dilution buffer as a control (E−) for 20 min. in room temperature. After incubation, CTCs were isolated according to the AdnaGen Prostate Cancer Select protocol.

All samples were further characterized according to the method described above (Characterization of the CTCs).

### Results

### Hyaluronidase Incubation Before CTC Isolation Increases the Number of Unique Genes Detected Compared to Standard CTC Isolation

All 15 patients included in this example displayed detection of at least 4 of the 35 genes and all were EPCAM positive (i.e. CTC-positive) in either the E+ or the E− sample. Two of the E− samples and one of the E+ samples did not have a detectable EPCAM-signal and were treated in the analysis as CTC negative in that sample. The detection level of EPCAM mRNA were approximately two times higher in E+ samples than in E−, with a mean value for “cycle of quantification” (Cq) in the qPCR analysis of 14.3 compared to 15.6 in E+ and E−, respectively. Thus, in this patient cohort, the EPCAM expression (interpreted as CTC amount) were statistically significantly higher in the E+ samples compared to the E− samples (p=0.044), indicating that the hyaluronidase incubation revealed CTCs not detected without the enzyme incubation.

The mean detection rate of the genes in the panel was 37% in controls (E−) and 43% after hyaluronidase incubation (E+) prior to characterization (table 4). Also in this example several genes were uniquely detected after hyaluronidase incubation (Table 4).

TABLE 4
The number of patients with detected expression for each
gene E+ and E− samples. The number of patients
with uniquely detected genes in E+ and E− respectively is shown.

| | | | | After enzymatic | | |
|---|---|---|---|---|---|---|
| | Control (E−) | | | incubation (E+) | | |
| | Patients | | | Patients | | |
| Gene | with | Detection | Unique | with | Detection | Unique |
| name | detection | rate | detection | detection | rate | detection |
| EPCAM | 13 | 87% | 1 | 14 | 93% | 2 |
| AGR2 | 10 | 67% | 0 | 11 | 73% | 1 |
| AHR | 2 | 13% | 1 | 3 | 20% | 2 |
| AKR1C3 | 8 | 53% | 1 | 10 | 67% | 4 |
| AKT2 | 6 | 40% | 3 | 7 | 47% | 5 |
| ALDH1A1 | 8 | 53% | 0 | 13 | 87% | 5 |
| AR | 6 | 40% | 0 | 7 | 47% | 1 |
| ARV7 | 5 | 33% | 1 | 4 | 27% | 0 |
| AURKA | 10 | 67% | 2 | 11 | 73% | 3 |
| BCL2 | 6 | 40% | 2 | 11 | 73% | 7 |
| CDH1 | 5 | 33% | 1 | 7 | 47% | 3 |
| CDH2 | 4 | 27% | 2 | 2 | 13% | 0 |
| DDR1 | 0 | 0% | 0 | 0 | 0% | 0 |
| EGFR | 2 | 13% | 1 | 2 | 13% | 1 |
| EMP2 | 7 | 47% | 1 | 8 | 53% | 2 |
| ESR1 | 0 | 0% | 0 | 0 | 0% | 0 |
| FOLH1 | 8 | 53% | 0 | 8 | 53% | 0 |
| UPA | 6 | 40% | 3 | 3 | 20% | 0 |
| HER2 | 5 | 33% | 0 | 5 | 33% | 0 |
| KLK3 | 5 | 33% | 1 | 8 | 53% | 4 |
| KRT19 | 8 | 53% | 2 | 7 | 47% | 1 |
| MDK | 5 | 33% | 0 | 7 | 47% | 2 |
| MET | 0 | 0% | 0 | 0 | 0% | 0 |
| POU5F1 | 12 | 80% | 2 | 11 | 73% | 1 |
| PSCA | 2 | 13% | 1 | 2 | 13% | 1 |
| PTCH1 | 2 | 13% | 1 | 1 | 7% | 0 |
| RUNX2 | 5 | 33% | 3 | 5 | 33% | 2 |
| SNAI1 | 1 | 7% | 0 | 2 | 13% | 1 |
| SPINK1 | 5 | 33% | 0 | 8 | 53% | 3 |
| SRD5A1 | 11 | 73% | 0 | 13 | 87% | 1 |
| TACSTD2 | 9 | 60% | 3 | 8 | 53% | 2 |
| TOP2A | 9 | 60% | 0 | 13 | 87% | 3 |
| TUBB3 | 2 | 13% | 0 | 2 | 13% | 0 |
| Twist1 | 4 | 27% | 1 | 5 | 33% | 2 |
| VEGFA | 6 | 40% | 1 | 8 | 53% | 2 |
| Mean | 5.63 | 37% | 0.97 | 6.46 | 43% | 1.74 |

To explore the difference of the CTC-populations isolated with and without hyaluronidase pre-incubation (E+ and E−) their expression patterns were compared. Most genes could be detected in both E+ and E− sample from the same patient ( 148/249; 60%), but in 27% ( 67/249) the gene was only detected in E+ CTCs and in 13% ( 34/249) the detection was only seen in the E− CTC (Table 5). The increase of uniquely detected genes in E+ samples was in average 36%, ranging from 0 to 80% among individual patients. Of the 14 men with detectable CTCs (detection of EPCAM) in E+ samples, 12 (86%) had uniquely detected genes in E+ samples indicating that hyaluronidase incubation may reveal CTCs with a different genotype compared to standard CTC isolation.

TABLE 5
Summary of the detected expression of genes in isolated CTC with (E+) and
without (E−) hyaluronidase incubation before CTC isolation for each patient.
The table displays the detection frequency of genes in E+ and E−
samples for each patient. A “0” represents no detection of the gene
neither before or after incubation, “2” represents detection in both
samples for that patient, “1” represents detection in only E+
CTC, and “−1” represents detection in only E− CTC.

| Gene | Patients | | | | | | | | | | | | | | |
|---|---|---|---|---|---|---|---|---|---|---|---|---|---|---|---|
| name | 1 | 2 | 3 | 4 | 5 | 6 | 7 | 8 | 9 | 10 | 11 | 12 | 13 | 14 | 15 |
| EPCAM | 2 | 1 | 2 | 1 | 2 | −1 | 2 | 2 | 2 | 2 | 2 | 2 | 2 | 2 | 2 |
| AGR2 | 2 | 1 | 2 | 0 | 2 | 0 | 1 | −2 | 2 | 2 | 2 | 2 | 2 | 0 | 0 |
| AHR | 1 | 0 | 0 | 0 | 0 | 0 | 2 | 0 | 0 | 0 | 0 | 1 | 0 | 0 | −1 |
| AKR1C3 | 2 | 1 | 2 | −1 | 2 | −1 | 2 | 0 | 2 | 1 | 0 | 0 | 1 | 1 | 2 |
| AKT2 | 1 | 0 | 2 | −1 | 1 | 0 | 2 | 0 | 1 | −1 | −1 | 1 | 0 | 1 | −1 |
| ALDH1A1 | 2 | 1 | 2 | 2 | 1 | 2 | 0 | 0 | 2 | 0 | 1 | 1 | 1 | 1 | −2 |
| AR | 2 | 0 | 0 | 0 | 2 | 0 | 2 | 0 | 2 | 0 | 1 | 2 | 0 | 0 | 0 |
| ARV7 | 2 | 0 | 2 | 0 | 2 | 0 | −1 | 0 | 2 | 0 | 0 | 0 | 0 | 0 | 0 |
| AURKA | 2 | 2 | 2 | 1 | 2 | 0 | 2 | 2 | 2 | 0 | −1 | 2 | 1 | 1 | −1 |
| BCL2 | 2 | 1 | 2 | 1 | 1 | 0 | 1 | 0 | −1 | 1 | 2 | 1 | 0 | 2 | 1 |
| CDH1 | 2 | 0 | 2 | 0 | 2 | 0 | 1 | 0 | 2 | 0 | 1 | 1 | 0 | 0 | 0 |
| CDH2 | −1 | 0 | 0 | 0 | 0 | 0 | 2 | 0 | 2 | 0 | 0 | 0 | 0 | 0 | −1 |
| DDR1 | 0 | 0 | 0 | 0 | 0 | 0 | 0 | 0 | 0 | 0 | 0 | 0 | 0 | 0 | 0 |
| EGFR | 0 | 0 | 2 | 0 | 1 | 0 | 0 | 0 | −1 | 0 | 0 | 0 | 0 | 0 | 0 |
| EMP2 | 2 | 0 | 2 | 0 | 2 | 0 | 2 | 0 | 2 | −1 | 1 | 2 | 1 | 0 | 0 |
| ESR1 | 0 | 0 | 0 | 0 | 0 | 0 | 0 | 0 | 0 | 0 | 0 | 0 | 0 | 0 | 0 |
| FOLH1 | 2 | 0 | 2 | 0 | 2 | 2 | 2 | 0 | 2 | 2 | 2 | 0 | 0 | 0 | 0 |
| UPA | −1 | 0 | 2 | 0 | 0 | −1 | 2 | 0 | 2 | 0 | 0 | 0 | 0 | 0 | −1 |
| HER2 | 2 | 0 | 2 | 0 | 2 | 0 | 0 | 0 | 0 | 0 | 2 | 2 | 0 | 0 | 0 |
| KLK3 | 2 | 0 | 2 | 0 | 1 | 0 | 2 | 0 | 2 | −1 | 1 | 1 | 1 | 0 | 0 |
| KRT19 | 2 | 0 | 2 | 0 | 2 | 0 | 2 | 0 | 2 | −1 | 1 | 2 | −1 | 0 | 0 |
| MDK | 1 | 0 | 2 | 0 | 2 | 0 | 2 | 0 | 2 | 0 | 1 | 2 | 0 | 0 | 0 |
| MET | 0 | 0 | 0 | 0 | 0 | 0 | 0 | 0 | 0 | 0 | 0 | 0 | 0 | 0 | 0 |
| POU5F1 | −1 | 1 | 2 | 0 | 2 | 2 | 2 | 1 | 2 | −1 | 2 | 0 | 2 | 2 | 2 |
| PSCA | 1 | 0 | 2 | 0 | 0 | 0 | −1 | 0 | 0 | 0 | 0 | 0 | 0 | 0 | 0 |
| PTCH1 | −1 | 0 | 2 | 0 | 0 | 0 | 0 | 0 | 0 | 0 | 0 | 0 | 0 | 0 | 0 |
| RUNX2 | 2 | 1 | −1 | 0 | 1 | 1 | 2 | 0 | −1 | 0 | 0 | 0 | 0 | 0 | −1 |
| SNAI1 | 0 | 0 | 2 | 0 | 0 | 0 | 1 | 0 | 0 | 0 | 0 | 0 | 0 | 0 | 0 |
| SPINK1 | 0 | 1 | 2 | 1 | 1 | 0 | 0 | 0 | 2 | 0 | 0 | −2 | 2 | 0 | 0 |
| SRD5A1 | 2 | 2 | 2 | 0 | 2 | 1 | 2 | 0 | 2 | 0 | 2 | 2 | 2 | 2 | 2 |
| TACSTD2 | 2 | 0 | 0 | 0 | 2 | −1 | 2 | 0 | −1 | 2 | 1 | 1 | 2 | 0 | −1 |
| TOP2A | 2 | 2 | 2 | 1 | 2 | 1 | 2 | 0 | 0 | 0 | 1 | 2 | 1 | 2 | 2 |
| TUBB3 | 0 | 0 | 2 | 0 | 0 | 0 | 0 | 0 | 2 | 0 | 0 | 0 | 0 | 0 | 0 |
| TWIST1 | 0 | 0 | 2 | 0 | 2 | 0 | 1 | 0 | 2 | 0 | 0 | 1 | −1 | 0 | 0 |
| VEGFA | 2 | 0 | 0 | 0 | 2 | 0 | 0 | 0 | 2 | 1 | 1 | 1 | 0 | 0 | −1 |

For each patient the median number of detected genes in E− and E+ samples was 10 (ranging from 4 to 29) and 13,5 (ranging from 4 to 28) respectively, also indicating that more genes were found after hyaluronidase incubation.

The increase of uniquely detected genes in E+ samples was in average 36%, ranging from 0 to 80% among patients. Of the 14 men with detectable CTCs (detection of EpCAM) in E+ samples, 12 (86%) had uniquely detected genes in E+ samples compared to E− samples. Of the 13 patients with detectable CTCs in E− samples, 9 (69%) had uniquely identified genes in the control sample (E−), not detected in the E+ sample from the same patient. However, in 60% of these cases, other genes were uniquely detected in the E+ samples.

### Hyaluronidase Incubation Before CTC Isolation Results in Detection of More Genes Encoding Drug Targets Compared to Standard Method

For 19 of the genes, detection was more common in E+ samples, while the corresponding number for control samples was 8 genes (FIG. 3). The genes most often uniquely detected after hyaluronidase incubation (identified uniquely in E+ samples in at least 3 (20%) of the 15 patients) were AKR1C3, AKT2, ALDH, AURKA, BCL2, CDH1, KLK3, SPINK1 and TOP2A (FIG. 3). All of these genes are potential treatment predictive markers or therapeutic targets, and up to five of their respective FDA approved drugs (if available) are listed in Table 6.

TABLE 6
List of genes uniquely detected in E+ samples in at least three
patients and corresponding FDA approved drugs for these targets.

| Gene | FDA Approved drugs with inhibition of the gene |
|---|---|
| name | pathway (max 5 drugs included) |
| AKR1C3 | Doxorubicin, Docetaxel, Exemestane and Doxil |
| AKT2 | Paclitaxel, Anastrozole, Carboplatin, Everolimus, |
| | Sirolimus |
| ALDH1A1 | Disulfiram and Guanidine |
| AURKA | Adenosine triphosphate |
| BCL2 | Docetaxel, Paclitaxel, Carboplatin, Rasagiline, |
| | Cisplatin |
| CDH1 | — |
| KLK3 | (PSA, the most used marker for prostate cancer |
| | follow up) |
| SPINK1 | — |
| TOP2A | Etoposide, Doxorubicin, Tenoposide, Daunorubicin |
| | and Epirubicin |

### CONCLUSIONS

These non-limiting examples reveal the novel findings that by adding hyaluronidase to the blood sample prior to two different standard methods of CTC isolation we could increase the number of CTCs detected. After incubation, we could also identify a higher number of expressed genes related to prostate cancer, which may indicate that a phenotypically different cell population was detected only after hyaluronidase incubation. A specific result from example 2 and 3 is that AR-V7, which has been shown to be a strong treatment predictive marker for prostate cancer therapy, was only detected after hyaluronidase incubation in two of the five patients. By treating with hyaluronidase before the standard isolation of CTC we could detect up to 80% more genes per patient (36% in mean) and the invention may therefore increase the detection of targetable markers or other treatment predictive markers for a given patient. These results strengthen the clinical relevance of the method described in this application.

1. A method of extracting circulating tumor cells (CTCs) from a blood sample from a human subject, comprising the steps of:
a. adding a hyaluronan degrading enzyme to a blood sample from a subject in order to remove hyaluronan from the surface of said CTCs;
b. bringing a CTC-capturing agent in contact with said CTCs; and
c. separating captured CTCs from said blood sample, wherein said hyaluronan degrading enzyme is a hyaluronidase.

2. The method according to claim 1, wherein said hyaluronidase is hyaluronate glycanohydrolase (EC 3.2.1.35).

3. The method according to claim 1, wherein the CTC-capturing agent comprises an antigen binding moiety, said moiety having affinity for a membrane-bound antigen of said CTCs.

4. The method according to claim 3, wherein said antigen binding moiety is immobilized on a magnetic bead.

5. The method according to claim 3, wherein said membrane-bound antigen is selected from epithelial cell adhesion molecule (EpCAM) and human epidermal growth factor receptor 2 (HER2).

6. The method according to claim 3, wherein said membrane-bound antigen is epithelial cell adhesion molecule (EpCAM).

7. The method according to claim 1, wherein at least some of said captured CTCs belong to a sub-population of CTCs.

8. The method according to claim 7, wherein said sub-population is not detectable without the addition of a hyaluronan degrading enzyme.

9. The method according to claim 1, wherein said sub-population of CTCs are of an especially aggressive phenotype.

10. The method according to claim 1, wherein the captured CTCs to a higher extent expresses detectable levels of genes selected from AR-V7, AKR1C3, AKT2, ALDH1A1, AURKA, BCL2, CDH1, KLK3, SPINK1 and TOP2A.

11. The method according to claim 1, wherein the human subject has, or is suspected of having, cancer.

12. The method according to claim 11, wherein the cancer is selected from breast cancer, colorectal cancer, ovarian cancer, bladder cancer, melanoma and prostate cancer.

13. The method according to claim 1, wherein the subject has or is suspected of having prostate cancer.

14. A method of i) diagnosing cancer in a human subject, or ii. predicting the outcome of cancer therapy in a subject, comprising performing the method according to claim 1, and:
d. analyzing the extracted CTCs.

15. A method of finding a target for therapy, comprising
performing the method according to claim 1;
characterizing the extracted CTCs with regard to expressed genes; and
selecting at least one therapeutic target from genes expressed in the extracted CTCs.

16. A method of treatment of a cancer in a human subject, said method comprising extracting circulating tumor cells according to claim 1, and
d. analyzing the extracted CTCs;
e. selecting a therapeutic agent based on the result of the analysis in d; and
f. administering a therapeutically effective amount of the therapeutic agent to a human subject in need thereof,
wherein analyzing is characterizing the CTCs with regard to gene expression, wherein the genes expressed correspond to potential therapeutic targets selected from AHR, AR, AKR1C3, AKT2, ALDH1A1, AURKA, BCL2, CDH1, SPINK1, TOP2A and VEGFA.

17. A kit for enhancing the extraction circulating tumor cells (CTCs) from a blood sample from a human subject, said kit comprising
a hyaluronan degrading enzyme;
a buffer with a pH of 5-9; and
instructions to use the kit with a CTC-capturing agent,
wherein said hyaluronan degrading enzyme is a hyaluronidase.

18. The kit according to claim 17, wherein said buffer is a phosphate buffer with a pH of 6-8.

19. The kit according to claim 17, wherein said kit further comprises a CTC-capturing agent.
